(19) United States
(12) Patent Application Publication
(10) Pub. No.: US 2022/0387160 A1
(43) Pub. Date: Dec. 8, 2022

(54) ADHESIVE SYSTEM FOR ROUGH SURFACES

(71) Applicant: Leibniz-lnstitut für Neue Materialien gemeinnützige GmbH, Saarbrücken (DE)

(72) Inventors: Eduard Arzt, Saarbrücken (DE); Klaus Kruttwig, Heidelberg (DE); Silviya Boyadzhieva, Saarbrücken (DE); René Hensel, Aalen (DE); Bernhard Schick, Hofbieber (DE); Gentiana Wenzel, Homburg (DE)

(21) Appl. No.: 17/775,972

(22) PCT Filed: Nov. 6, 2020
(86) PCT No.: PCT/EP2020/081269

§ 371 Date: May 11, 2022

(30) Foreign Application Priority Data
Nov. 15, 2019 (DE) 10 2019 130 988.3

(51) Int. Cl. A61F 2/00

(52) U.S. Cl. CPC A61F 2/0077; A61F 2002/183

(57) ABSTRACT

A device having a structured coating for adhering to rough, in particular, biological surfaces, includes a carrier layer, wherein a plurality of protrusions is arranged on the carrier layer, which protrusions each comprise at least one stem having an end face pointing away from the surface, and wherein a further layer is arranged at least on the end face, wherein the layer has a lower modulus of elasticity and is in the form of a film that interconnects the protrusions. The film can also be in the form of a removable film.

### FIELD OF THE INVENTION

The invention relates to a device having a structured coating, particularly for adhesion to rough surfaces, especially biological surfaces, the skin surface, for example, such as eardrums, for example.

Adhesion to rough surfaces frequently presents problems. There are many adhesives which in the biological sector in particular exhibit only inadequate properties. At the same time there is also the problem that adhesives lack sufficient compatibility with biological processes, such as wound healing, for example.

One alternative is offered by dry-adhesive surfaces, such as gecko structures, for example, which are able to exhibit adhesion even to rough surfaces without mediation by adhesives.

On skin surfaces in particular, adhesion is not simple, as these surfaces are both rough and soft. The surfaces also often are curved rather than being planar. At the same time the sticking system ought to be removable again without residues. A sticking system must therefore first be flexible, but also stick with sufficient strength.

Another field of application for a sticking system is that of drum perforations. Eardrum perforations are a frequently occurring problem, which can lead to loss of hearing or to frequently recurring infections. A frequent cause of eardrum perforations may be middle-ear inflammation, trauma and postoperative complications. Distinctions may be made fundamentally between acute (relatively minor) perforations, which in the majority of cases close up spontaneously, and major or chronic perforations. These major perforations require surgical care in the form of myringoplasty or tympanoplasty, where success rates are high but as well as the surgical risk there is also the danger of residual perforation. In the case of tympanoplasty, moreover, autologous tissue is transplanted, and must additionally be withdrawn. One of the main problems in the regeneration of eardrum injuries is the lack of a backing layer for the migration of epithelial cells, and the formation of a trilamellar membrane. As “support platforms” it is possible generally to use either transplanted tissues or polymers, whose function may then be improved by the use of biomolecules. Polymers which can be used include, among others, gelatin, silk fibroin, chitosan, alginates or polyglycerol sebacates. A current review of results in the use of these polymers and of various growth factors is found in the review by Hong et al. Int. J. Pediatr. Otorhinolaryngol. 77, 3-12 (2013). Although many of the polymers used lead to outstanding results in terms of closing the perforation, there are significant differences in the morphology of the tissue.

Hamed Shahsavan et al. Soft Mater 2012, 8, 8281 “Biologically inspired enhancement of pressure-sensitive adhesives using a thin film-terminated fibrillar interface”, Hamed Shahsavan et al. Macromolecules 2014, 47, 353-364 and Drotlef et al. Integrative and Comparative Biology, 2019, 1-9 describe various systems with film-terminated microstructures. They use pillars with a high modulus of elasticity of around 2.7 MPa (Sylgard 184) for their structures.

### OBJECT

The object of the invention is that of specifying a device having a structured coating which exhibits adhesion particularly to rough and/or to biological surfaces and avoids the disadvantages of the prior art.

### ACHIEVEMENT

This object is achieved by the inventions having the features of the independent claims. Advantageous developments of the inventions are characterized in the dependent claims. The wording of all of the claims is hereby made part of this description by reference. The inventions also embrace all rational combinations and more particularly all stated combinations of independent and/or dependent claims.

The object is achieved by a device having a structured coating, the device comprising a backing layer bearing a multiplicity of protrusions (pillars) which comprise at least in each case a stem having an end face pointing away from the surface, wherein the end faces bear at least one further layer which is configured as a film, this layer comprising as surface at least one layer which has a lower modulus of elasticity than the respective protrusion.

This layer configured as a film connects the various protrusions. The film itself may comprise different layers, with the outermost layer, which forms the surface of the film on the side facing away from the protrusions, having a lower modulus of elasticity than the protrusions. This layer forms the contact to the surface to which the device is applied.

In a perpendicular direction, therefore, the device, at the position of a protrusion, comprises, starting from the backing layer, at least two regions with different moduli of elasticity, these regions being at least the protrusion and the further layer disposed thereon. This further layer and the end face of a protrusion form an interface between two regions with different moduli of elasticity. Depending on the production process, the interfaces may also comprise thin layers of connection assistants.

Within a region the modulus of elasticity is preferably constant.

A protrusion itself may also have further regions which have a different modulus of elasticity. In this case the lower modulus of elasticity of the further layer always relates to the region of the protrusion having the highest modulus of elasticity.

The further layer has a lower modulus of elasticity than the protrusion bearing it. The effect of this construction is that the outermost layer of the device is particularly soft. As a result, the layer is more elastic and is able more effectively to conform to rough and/or soft surfaces as well.

In the case of a device which is very soft overall, this device is also able to conform very well to curved surfaces.

The devices of the invention exhibit particularly good adhesion to surfaces having a roughness depth R_{z} of at least 30 μm, preferably at least 40 μm, especially in direct comparison to smooth surfaces having a roughness depth of 0.1 μm. The device therefore exhibits particularly good adhesion to surfaces having a roughness depth Rz of up to 100 μm, more particularly up to 80 μm, especially up to 70 μm.

In a further embodiment of the invention, the interface between further layer and end face is parallel to the surface of the further layer relative to the respective protrusion.

In one embodiment of the invention, the ratio of the minimum perpendicular thickness of the further layer above the protrusion in relation to the height of the protrusion is less than 3, preferably less than 1, more particularly less than 0.5, more particularly less than 0.3. As a result the protrusions below the layer have a particularly strong effect on the adhesion. The optimum ratio may also depend on the ratio of the moduli of elasticity, and also on the geometry of the interface.

The advantageous parameters for modulus of elasticity, size ratio and geometry of the interface may be determined by simulations and measurements.

In one preferred embodiment of the invention, the protrusions on the backing layer have a columnar configuration. This means that the protrusions preferably are protrusions configured perpendicularly to the backing layer and having a stem and an end face, it being possible for the stem and the end face to have any desired cross section (for example, circular, oval, rectangular, square, rhomboid, hexagonal, pentagonal, etc.).

The protrusions are preferably configured such that the perpendicular projection of the end face onto the base area of the protrusion forms an overlap area with the base area, with the overlap area and the projection of the overlap area onto the end face generating a body which lies completely within the protrusion. In one preferred embodiment of the invention, the overlap area comprises at least 50% of the base area, preferably at least 70% of the base area, and more preferably the overlap area comprises the entire base area. The protrusions are therefore preferably not inclined, but may be.

In one preferred embodiment, the end face is oriented parallel to the base area and to the surface. If the end faces are not oriented parallel to the surface and therefore have different perpendicular heights, the perpendicular height of the protrusion is considered to be the mean perpendicular height of the end face.

In one preferred embodiment of the invention, the stem of the protrusion, based on its mean diameter, has an aspect ratio of height to diameter of 1 to 100, preferably of 1 to 10, more preferably of 1.5 to 5.

In one embodiment, the aspect ratio is greater than 1, preferably at least 1.5, preferably at least 2, preferably 1.5 to 15, more preferably 2 to 10.

The mean diameter here is understood as the diameter of the circle having the same area as the corresponding cross section of the protrusion, averaged over the entire height of the protrusion.

In another embodiment of the invention, the ratio of the height of a protrusion to the diameter at a particular height over the entire height of the protrusion is always 1 to 100, preferably 1 to 10, more preferably 1.5 to 5. In one embodiment, this aspect ratio is at least 1, preferably 1 to 3. The diameter here is understood to be the diameter of the circle which has the same area as the corresponding cross section of the protrusion at the particular height.

The protrusions may have broadened end faces, known as “mushroom” structures. It is also possible for the further layer to project over the end face and so to form a “mushroom” structure.

In one preferred embodiment, the protrusions do not have any broadened end faces.

In one preferred embodiment, the perpendicular height of all the protrusions is in a range from 1 μm to 2 mm, preferably 10 μm to 1 mm, more particularly 10 μm to 500 μm, preferably in a range from 10 μm to 300 μm.

In one preferred embodiment, the total perpendicular thickness of the further layer, comprising all included layers above an end face, is in a range from 1 μm to 1 mm, preferably 1 μm to 500 μm, more particularly 1 μm to 300 μm, preferably in a range from 1 μm to 200 μm, more particularly in a range from 5 μm to 100 μm, especially 5 μm to 60 μm.

The further layer preferably has a perpendicular thickness within the above range or one of the preferred ranges, based on at least 50% of the projection of the base area of a protrusion onto the surface of the further layer. The thickness is preferably also the average thickness of the entire further layer over the entire device.

The smallest thickness of the further layer above a protrusion is preferably always less than the maximum perpendicular height of the protrusion.

In one preferred embodiment, the perpendicular thickness of the backing layer is in a range from 1 μm to 2 mm, preferably 20 μm to 500 μm, more particularly 20 μm to 150 μm. In one preferred embodiment, the thickness of the backing layer is 20 to 60 μm.

In one preferred embodiment, the base area corresponds in terms of the area to a circle having a diameter of between 0.1 μm and 5 mm, preferably 0.1 μm and 2 mm, especially preferably between 1 μm and 500 μm, very preferably between 1 μm and 100 μm. In one embodiment, the base area is a circle having a diameter of between 0.3 μm and 2 mm, preferably 1 μm and 100 μm.

The mean diameter of the stems is preferably between 0.1 μm and 5 mm, preferably 0.1 μm and 2 mm, especially preferably between 10 μm and 100 μm. The height and the mean diameter are preferably adapted in accordance with the preferred aspect ratio.

In one preferred embodiment, in the case of broadened end faces, the surface of the end face of a protrusion, or the surface of the further layer, is at least 1.01 times, preferably at least 1.5 times, the area of the base area of a protrusion. It may be greater, for example, by a factor of 1.01 to 20.

In another embodiment, the broadened end face is between 5% and 100% larger than the base area, more preferably between 10% and 50% of the base area.

In one preferred embodiment, the distance between two protrusions is less than 2 mm, more particularly less than 1 mm, especially less than 500 μm or less than 150 μm. The distance here is understood to be the shortest remove between two protrusions.

The protrusions preferably have a regular periodic arrangement.

In one preferred embodiment of the invention, the protrusions have a height of 5 up to 500 μm, preferably up to 400 μm. The further layer has a total perpendicular thickness, above the end faces, of 3 to 100 μm. The mean distance between the columnar protrusions is between 5 and 50 μm. The thickness of the backing layer is between 50 and 200 μm. The diameter, depending on the distance between the protrusions, is 5 to 100 μm. The protrusions are preferably arranged hexagonally. Very preferably the density of the protrusions is 10 000 to 1 000 000 protrusions/cm².

The total thickness of the device comprising the further layer, the protrusions and the backing layer is preferably between 50 μm and 500 μm. The thickness of the individual constituents is adapted correspondingly.

In one embodiment of the invention, the total thickness of the device is between 40 and 90 μm. With these thin devices it is preferred for the protrusions to occupy at least 30% of the total height of the device, preferably at least 40%.

The moduli of elasticity of all of the regions of the protrusion and of the further layers are preferably 40 kPa to 2.5 MPa. The modulus of elasticity of soft regions, i.e., in particular, of the further layer with lower modulus of elasticity, is preferably 40 kPa to 800 kPa, preferably 50 kPa to 500 kPa, more preferably 50 to 150 kPa. Independently of this, preferably, the modulus of elasticity of the regions having a high modulus of elasticity, e.g., of the protrusions and also, for example, the backing layer, is 1 MPa to 2.5 MPa, preferably 1.2 MPa to 2 MPa. Preferably, for all softer and harder regions, the moduli of elasticity are within the ranges specified above (measured using nanoindenter).

The ratio of the moduli of elasticity between the lowest modulus of elasticity and the region with the highest modulus of elasticity is preferably below 1:100, more particularly below 1:80, preferably below 1:70, independently thereof at least 1:2, preferably at least 1:3.

In one preferred embodiment, the modulus of elasticity of the protrusions and of the backing layer, and also, where appropriate, a region of the further layer, is 1 MPa to 2.5 MPa, preferably 1.2 MPa to 2 MPa, whereas for the regions with a lower modulus of elasticity, the modulus of elasticity is 40 kPa to 800 kPa, preferably 50 kPa to 500 kPa, more preferably 50 to 150 kPa (measured using nanointender).

The use of such a soft material for the protrusions and the backing layer permits the production of relatively thick but relatively elastic devices, which, while having adhesion values similar to those of stiffer structures, are nevertheless significantly more flexible. As a result of connection via a film, the protrusions are additionally stabilized. This prevents the soft protrusions from collapsing. At the same time, thicker devices can be produced more simply and handled more easily.

As a result of the stabilization by a film, the device as well is stabilized itself. This is important, for example, if the device is intended to withstand not only the adhesion but also tensile forces parallel to the contact face. For example, on application to wounds to be closed or to eardrum injuries. This additionally permits a reduction in the modulus of elasticity of the protrusions and of the backing layer, without loss of stability of the protrusions in particular.

In another embodiment, the ratios specified above describe the ratio of the moduli of elasticity of the further layer (soft) and of the protrusions (hard).

This layer as well is simple to keep clean, or sterile, since no dirt at all can collect in the interspaces. When used on the eardrum, in particular, an infection barrier with respect to microorganisms is constructed as a result. This “sealing”, furthermore, also leads to an improvement in hearing performance in the case of a perforated eardrum.

This gives the surface of the device in this embodiment a cohesive and unitary appearance. As a result it can also be modified more easily, to be adapted for applications. In that case a treatment of the surface has no effect on the structuring within the coating.

The surface can accordingly be treated or functionalized with known processes.

The interspaces between the protrusions within the device are preferably not filled in. It is also possible for the interspaces to be filled in between, with the material having a different modulus of elasticity from the protrusions and the backing layer.

The protrusions may consist of numerous different materials, preference being given to elastomers and particular preference to crosslinkable elastomers. The regions with a higher modulus of elasticity may also comprise thermosets.

The protrusions and also the further layer may therefore comprise the following materials:

epoxy- and/or silicone-based elastomers, polyurethanes, epoxy resins, acrylate systems, methacrylate systems, polyacrylates as homo- and copolymers, polymethacrylates as homo- and copolymers (PMMA, AMMA acrylonitrile/methyl methacrylate), polyurethane (meth)acrylates, silicones, silicone resins, rubber, such as R rubber, NR natural rubber, IR polyisoprene rubber, BR butadiene rubber, SBR styrene-butadiene rubber, CR chloropropene rubber, NBR nitrile rubber, M rubber (EPM ethene-propene rubber, EPDM ethylene-propylene rubber), unsaturated polyester resins, formaldehyde resins, vinyl ester resins, polyethylenes as homo- or copolymers, and also mixtures and copolymers of the aforesaid materials. Also preferred are elastomers which are approved for use in the packaging, pharmaceutical and food sector by the EU (in accordance with EU Reg. No. 10/2011 of 14.01.2011, published on 15 Jan. 2011) or FDA, or silicone-free, UV-curable resins from PVD and CVD process engineering. Polyurethane (meth)acrylates here stand for polyurethane methacrylates, polyurethane acrylates, and also mixtures and/or copolymers thereof.

The materials in question may also be hydrogels, based for example on polyurethanes, polyvinylpyrrolidone, polyethylene oxide, poly(2-acrylamido-2-methyl-1-propanesulfonic acid), silicones, polyacrylamides, hydroxylated polymethacrylates or starch.

Preference is given to epoxy- and/or silicone-based elastomers, polyurethane (meth)acrylates, polyurethanes, silicones, silicone resins (such as UV-curable PDMS), polyurethane (meth)acrylates, rubber (such as EPM, EPDM).

Particularly preferred are crosslinkable silicones such as, for example, polymers based on vinyl-terminated silicones.

Especially for the further layer which is in contact with the surface, preference among the materials identified above is given to the epoxy- and/or silicone-based elastomers, polyurethane (meth)acrylates, polyurethanes, silicones, silicone resins (such as UV-curable PDMS), polyurethane (meth)acrylates, rubber (such as EPM, EPDM), more particularly to crosslinkable silicones such as, for example, polymers based on vinyl-terminated silicones.

It is also possible to use the above-stated hydrogels or pressure-sensitive adhesives for the further layer.

In one preferred embodiment of the invention, the further layer comprises at least one layer having a relatively high modulus of elasticity (hard), preferably the modulus of elasticity of the protrusions, and also, thereupon, the layer having the lower modulus of elasticity. The lower layer (supporting layer) stabilizes the layer having the lower modulus of elasticity (adhesion layer). As a result it is possible to recruit particularly soft materials for this layer, without the layer sinking between the protrusions.

In this embodiment, the thickness of the supporting layer is between 1 and 100 μm and the thickness of the adhesion layer is between 5 and 100 μm; preferably the thickness of the supporting layer is between 1 and 50 μm and the thickness of the adhesion layer is between 10 and 50 μm; very preferably the supporting layer is between 1 and 20 μm thick and the adhesion layer is between 1 and 20 μm thick.

In another preferred embodiment of the invention, the further layer has only a relatively low modulus of elasticity (adhesion layer). In that case there is indeed a certain sinking of the layer between the protrusions, but conformance to rough surfaces is still very effectively possible owing to the high elasticity of the layer.

In this embodiment, the thickness of the further layer is between 5 and 100 μm, preferably between 10 and 50 μm.

In another embodiment, the surface of the further layer is treated. The properties of the surface may be influenced in this way. This may take place by physical treatment such as plasma treatment, preferably with Ar/O₂ plasma.

It is also possible for covalent or noncovalent bonds to be formed to additives on the surface, in order, for example, to achieve a certain compatibility with the cells. Preferred additives are those for supporting cell adhesion, such as poly-L-lysine, poly-L-ornithine, collagen or fibronectin, for example. Additives of these kinds are known from the cell culture field.

Especially in the context of use in the medical sector, it may also be advantageous, in at least part of the device, to house substances which are then slowly delivered. These substances may be, for example, medicinal products, such as antibiotics, or else auxiliaries for supporting cell adhesion or cell growth.

In another embodiment, the protrusions and the backing layer are made of the same material.

In another embodiment of the invention, the further layer having the lower modulus of elasticity is implemented so as to be detachable from the device, with preferably the entire further layer of the device being detachable. Detachable here means that in particular there are no covalent bonds between the detachable layer and the rest of the device, as for example between the protrusions and the further layer. The bonding is based only on noncovalent bonds.

In one preferred embodiment of the invention, the further layer, starting from the end faces, comprises a layer of low modulus of elasticity for bonding to the end faces, a supporting layer, and also the layer with lower modulus of elasticity, for adhesion to a surface.

The inner layer with lower modulus of elasticity serves for adhesion to the protrusions and is connected only by the adhesion forces. As a result it is possible for the part of the device having the protrusions to be separated off and used again.

Through contact with the surface, the outermost layer of the device is easily soiled and can therefore not be used again after detachment, in the case of medical applications, for example. If the further layer can simply be switched with this layer, the part of the device having the protrusions can be easily used again, just by applying a new further layer. A coated supporting layer is easier to produce than the part of the device having the protrusions.

In one preferred embodiment of the invention, the further layer is detachable and, starting from the protrusions, has the following construction: inner adhesion layer, supporting layer, and outer adhesion layer. The inner supporting layer serves to stabilize the detachable further layer, to prevent tearing during detachment. In that case the layer also has better handling qualities. The adhesion layer to the protrusions ensures adhesion of the further layer to the protrusions.

In this embodiment, the further layer has a total thickness of 50 to 300 μm, preferably 50 to 150 μm.

In this case the thickness of the inner adhesion layer is preferably from 5 to 100 μm, preferably 10 to 50 μm. Independently of this, the supporting layer has a thickness of 5 to 100 μm, preferably of 10 to 50 μm. Independently of this, the outer adhesion layer has a thickness of 10 to 50 μm.

In one preferred embodiment, the modulus of elasticity of the supporting layer is 1 MPa to 2.5 MPa, preferably 1.2 MPa to 2 MPa, while the adhesion layers have a modulus of elasticity of 40 kPa to 800 kPa, preferably 50 kPa to 500 kPa, more preferably 50 to 150 kPa.

The dimensions of the microstructure correspond to the details above for the other embodiments.

For this embodiment with the detachable further layer, it also permits the use of a microstructure made of a relatively stiff material and the attainment likewise of improved adhesion.

In the case of this embodiment, the modulus of elasticity of the protrusions and of the backing layer is preferably 1 MPa to 4 MPa, preferably 1 MPa to 3 MPa, more preferably 1 MPa to 2.5 MPa, especially preferably 1.2 MPa to 2 MPa.

In another embodiment, the device also comprises further layers, which are optionally detachable. Accordingly the surfaces may be protected by detachable films prior to use. Further stabilizing layers may also be disposed on the backing layer.

The backing layer preferably has a thickness lower than the maximum height of the protrusions it bears.

Since the backing layer, if consisting of the same material as the protrusions, comprises a material having a relatively high modulus of elasticity, the thickness of the backing layer can also be used to influence the elasticity of the overall device.

The device of the invention is configured preferably for adhesion on soft substrates.

The device of the invention is configured more particularly for adhesion to biological tissues. For this purpose it may be implemented, for example, as a film. It may also be implemented in combination with the devices to be affixed. These may be, for example, dressing materials, or else electrodes or other medical devices such as implants, more particularly implants which are not to be anchored permanently to bone, or soft implants. These may be, for example, iris implants. The invention therefore also relates to an implant comprising a device of the invention, on at least part of the surface of the implant, for example.

The invention further relates to the use of an above-specified device for adhesion to biological tissues. These may be any desired tissues, such as skin or else internal tissues such as organ surfaces, surfaces of wounds, or eardrums. When mounted on skin, this may be healthy or damaged tissue. The device may be used for securing, such as for sensors, dressings, patches, infusions or the like, for example. The device may alternatively be applied to damaged tissue, such as superficial injuries such as wounds, burns, pressure points, chronic wounds or the like. The device permits the combination of a highly compatible surface with simultaneous adhesion on the biological tissue. Accordingly the device may also serve as a growth substrate for cell culturing or for the new tissue to be formed. As a result of the inner open structure of the device, it is also possible for liquid to drain off or air to circulate.

Treatment of Eardrum Perforations

As a result of the adhesion of the device, the device sticks very well to the surface of the eardrum and even makes it possible to be applied under stress, or to apply stress. Because of its structure, it also sticks on the surrounding tissues and not only on the eardrum. The device thus configured may optionally comprise different regions differing in their adhesion. This may be accomplished, for example, by way of the material, the layer thickness of the further layer, or else, simply, by the distribution of the protrusions within the device.

The device, implemented advantageously as a film, therefore comprises at least the backing layer with the protrusions, with the further layer applied to these protrusions. As a result of implementation as a film, the device can easily be trimmed to the desired size. This may even take place by the person carrying out the treatment, such as the doctor.

As a result of its inner structuring, the device sticks well to the tissue to which it is applied. This may be the tissue surrounding the eardrum as well as the eardrum itself. No liquid constituent, which can flow into the ear, is needed in order to apply the device.

Depending on the materials used, the device may also be transparent, thus permitting investigation of the condition of the tissue below the device without detachment, in order to determine the healing, for example.

The device can easily be detached again.

Prior to deployment, the device may also be treated physically or chemically, preferably for sterilization. This may be, for example, an autoclaving process, by hot air sterilization for example or steam sterilization at 50 to 200° C., more particularly 100 to 150° C., under a pressure of 1 to 5 bar, for 5 minutes to 3 hours. In the course of such autoclaving (121° C., 2 bar, 20 minutes) it has not been possible to observe any significant alteration of the sticking stress.

Further methods of sterilization are, for example, gamma rays or ethylene oxide sterilization (ETO).

In another embodiment, the surface may be treated, for example, with poly-L-lysine, poly-L-ornithine, collagen, fibronectin, gelatin, laminins, keratin, tenascin or perlecan. Such additives are known from the cell culture sector.

The invention further relates to a process for producing an embodiment of the device of the invention.

Individual process steps are described in more detail below. The steps need not necessarily be carried out in the order stated, and the process to be outlined may also comprise further steps not stated.

To this end, in a first step, a template is provided for modeling the multiplicity of protrusions.

The material for the protrusions is introduced into this template, preferably as a liquid. The material may optionally also be already at least partially cured.

The material for the backing layer, i.e., the surface bearing the protrusions, is then applied to the template and cured. With particular preference this is the same material as for the stems of the protrusions, and so the backing layer and the stems are also produced in one step, by the direct introduction, for example, of a relatively large amount of material.

In a subsequent step, the backing layer and the protrusions are parted from the template.

It may be necessary to render the template inert prior to filling, using fluorosilanes, for example.

It may also be necessary to align the protrusions, by mechanical action such as stroking or brushing, for example.

The material for one of the further layers is also distributed on a surface, by spincoating, for example. Thereafter this layer is cured. This may be repeated multiply using different materials.

For attachment to the protrusions, curable material is applied to the topmost layer and distributed, by spincoating, for example. The microstructure with the protrusions is then placed onto this layer in such a way that the end faces make contact with the layer. Thereafter the entire device is cured. As a result, the further layer is firmly connected to the protrusions. The device is subsequently parted from the surface.

Depending on material and structure, it may be necessary to carry out a plasma treatment, preferably oxygen plasma or air plasma, between the application of the various materials. This makes it possible to minimize the influencing of the different layers in the course of curing. The sticking is improved as well.

It may also be necessary to subject the end faces of the microstructure to a plasma treatment prior to the placement. This is the case when, for example, the contact area of the microstructure is particularly small.

Problems during detachment may occur especially if the first layer applied is very soft.

In another embodiment, a layer of a material possessing a solubility different from that of the materials of the cured device is applied to a substrate, allowing it to be selectively dissolved.

Then—as described above—the further layers and the microstructure are applied to this auxiliary layer. Thereafter the auxiliary layer is selectively dissolved, and the resulting device is thus parted from the substrate. The material of the auxiliary layer is preferably water-soluble, by treatment in ultrasound, for example. Preferred materials for the auxiliary layer are water-soluble polymers such as polyvinyl acetate.

In this process, therefore, first the auxiliary layer is applied to a substrate and optionally cured. Thereafter the material for the topmost layer of the device, the adhesion layer, is applied to this auxiliary layer and cured. After that, depending on the nature of the device being produced, further layers are applied. These may be further soft layers or else supporting layers. The layers may in each case be cured. Thereafter the microstructure is applied. It may be necessary, as described above, for an uncured layer to be applied beforehand, this layer being cured only after the application of the microstructure. After that, the auxiliary layer is selectively dissolved and the device is detached. It may be necessary to clean the surface as well in order to remove remnants of the auxiliary layer.

In one embodiment of the invention, instead of an auxiliary layer, a particularly readily detachable material is used as substrate for the first layer. Preference in this case is given to materials having a coating of fluorinated silicones or fluorinated silanes, examples being release liners. The material in question may comprise, for example, a film having such a coating.

The release liner ought to have an extremely smooth surface, since any unevenness is reproduced on the topmost layer.

Further details and features are apparent from the following description of preferred exemplary embodiments in conjunction with the dependent claims. In these contexts the respective features may be realized on their own or as a plurality in combination with one another. The possibilities for achieving the object are not confined to the exemplary embodiments. For example, range indications always encompass all—unstated—intermediate values and all conceivable sub-intervals.

The exemplary embodiments are represented schematically in the figures. Identical reference numerals in the individual figures here designate identical or functionally identical elements or elements which correspond to one another in terms of their functions. Specifically:

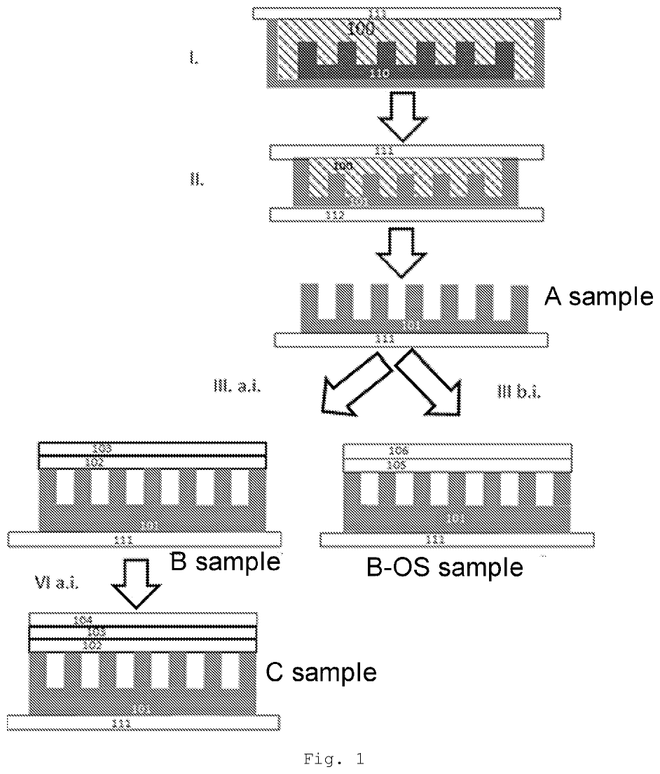

FIG. 1 shows an overview of the operation of producing the film-terminated sticking structures;

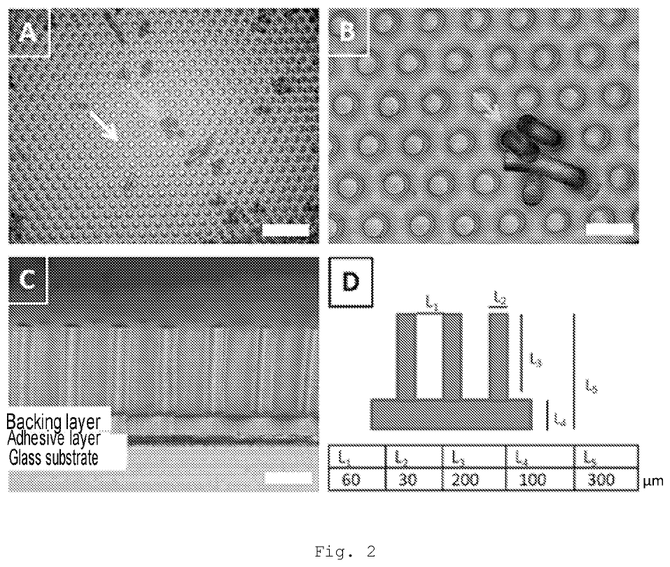

FIG. 2 shows an overview of the A sample in plan view at low magnification (A), the bottom arrow showing an upright pillar, the orange arrow a number of collapsed pillars; overview of the A sample in plan view at greater magnification (B), the collapsed pillars being shown from close up (top arrow); overview of the cross section of the A sample at high magnification (C) with dissolved layers of the substrate (adhesive layer and glass substrate) serving only for securement; schematic overview of the A sample, with MDX-4 being marked in gray; (D) with indication of size order, all lengths being in μm. The scale is 500 μm for A and 100 μm for B and C;

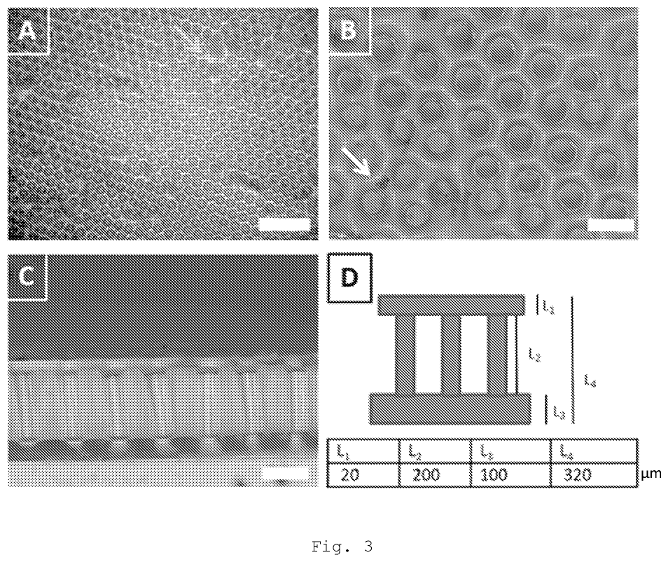

FIG. 3 shows an overview of the B sample in plan view at low magnification (A)—the arrow points to a vacancy caused by collapsed pillars; overview of the B sample in plan view at greater magnification (B)—the arrow points to unevennesses and impurities on the surface; overview of the cross section of the B sample at high magnification (C); schematic overview of the B sample, with MDX-4 being marked in gray; (D) with indication of size order, all lengths being in μm. The scale is 500 μm for A and 100 μm for B and C;

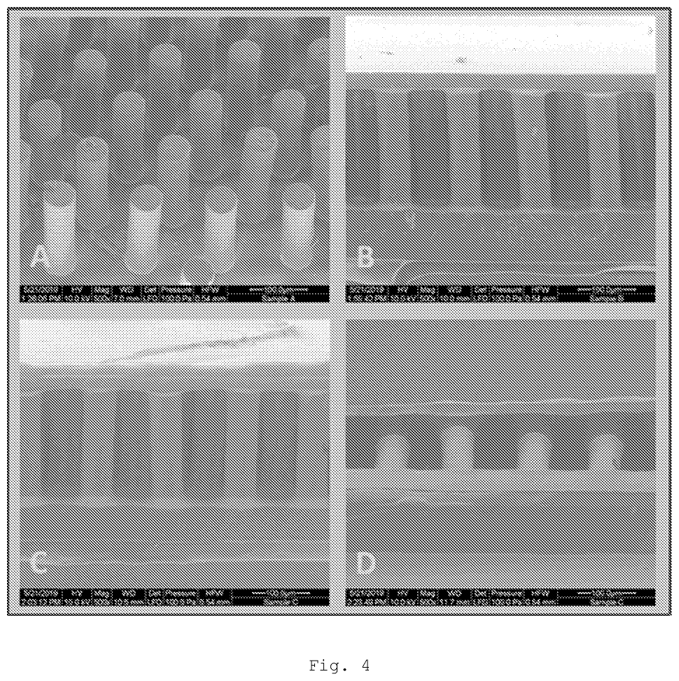

FIG. 4 shows SEM micrographs of the samples: the A sample: only the microstructured part is depicted (A). B) shows the B sample, where the terminal film, composed of the same material as the microstructured part, has been applied as supporting layer (B). The * points to the terminal layer. C) shows the C sample, following application of the soft, skin-adhering layer (C). The * points to the boundary layer between the two layers. D) allows a view of the bottom side of the terminal layer (D);

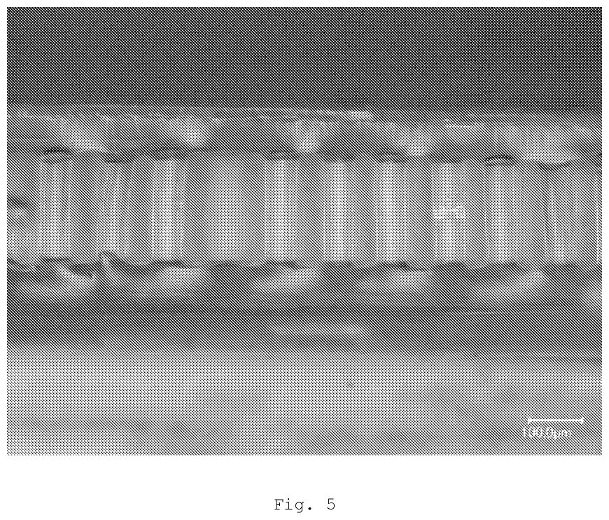

FIG. 5 shows a cross section of the C sample;

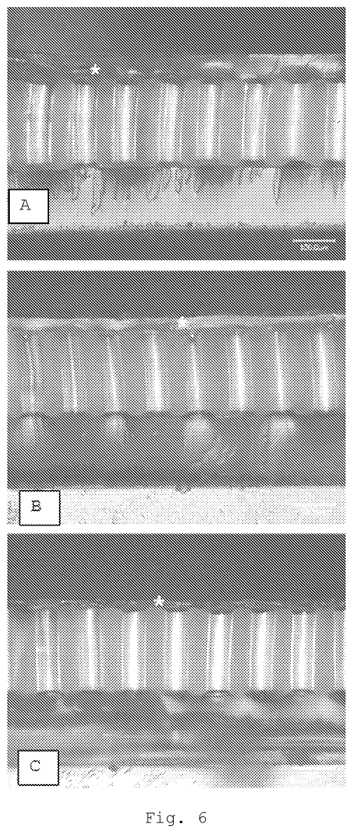

FIG. 6 shows cross sections of different B samples: the layer thickness of the terminating layer may be adjusted here in a defined way by means of spincoating. A spincoating speed of 800 rpm (A) results in a layer thickness of 60.5 μm, 2000 rpm (B)=31.3 μm, 9000 rpm (C)=12.2 μm. The layer thickness may also be reduced further by the addition of a solvent to the polymer;

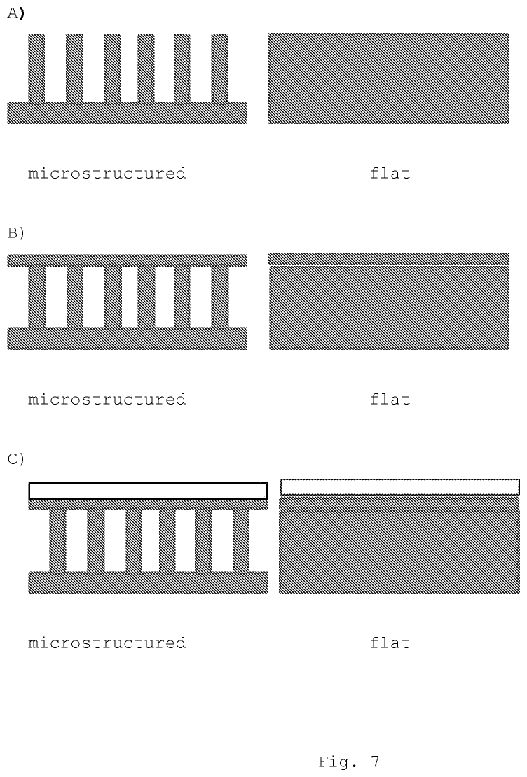

FIG. 7 shows various microstructured samples and flat reference samples with comparable thickness and construction; A) A sample with backing layer and microstructure, and A reference sample; B) B sample with backing layer, microstructure and supporting layer, and B reference sample with base and supporting layer; C) C sample with backing layer, microstructure, supporting layer and “bonding layer”, and C reference sample with base, supporting layer and “bonding layer”, in each case from bottom to top;

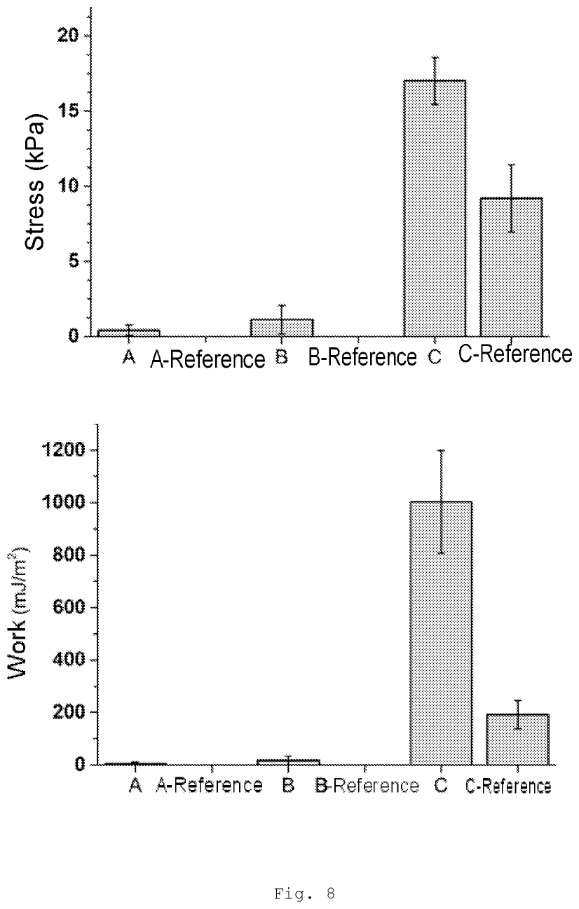

FIG. 8 shows stress and detachment energy (work) from the samples from FIG. 7 and table 1 (holding time 1 second);

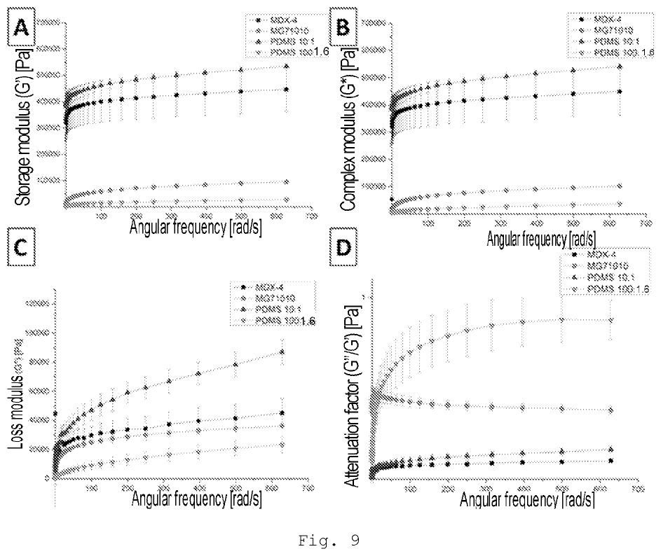

FIG. 9 shows rheology measurements for various samples;

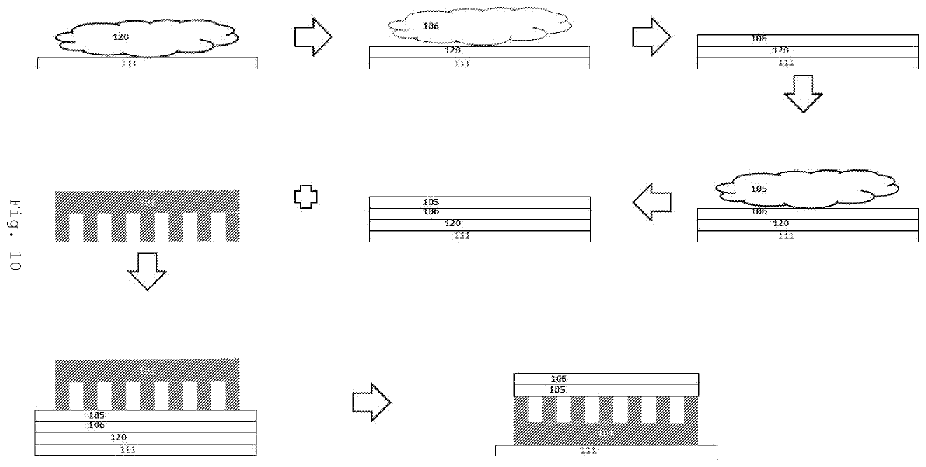

FIG. 10 shows production of film-terminated pillars without supporting layer;

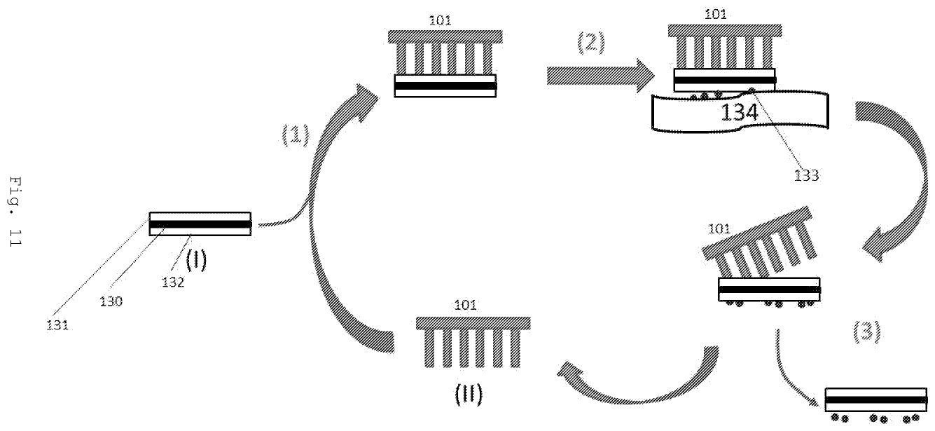

FIG. 11 shows a schematic representation for the use of the sticking system with partable film;

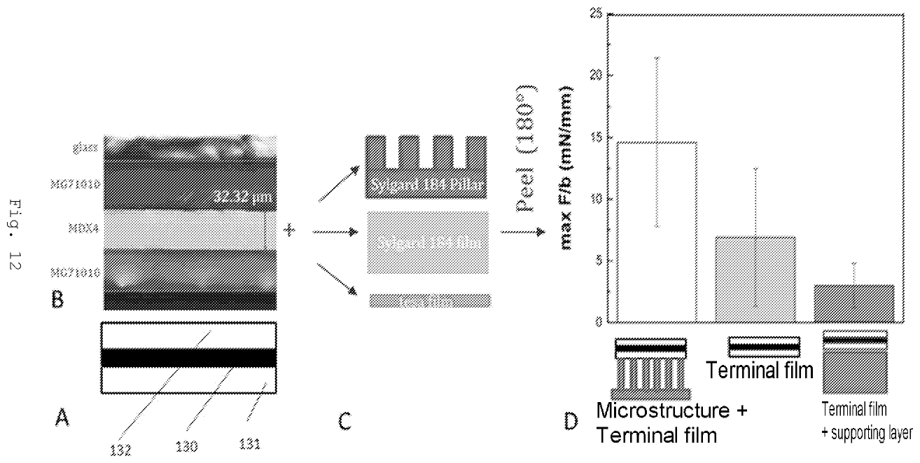

FIG. 12 shows an exemplary embodiment of a sticking system with partable film;

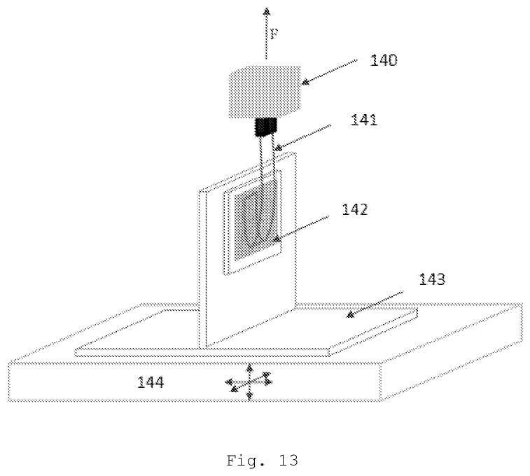

FIG. 13 shows a schematic representation of the peel measurement;

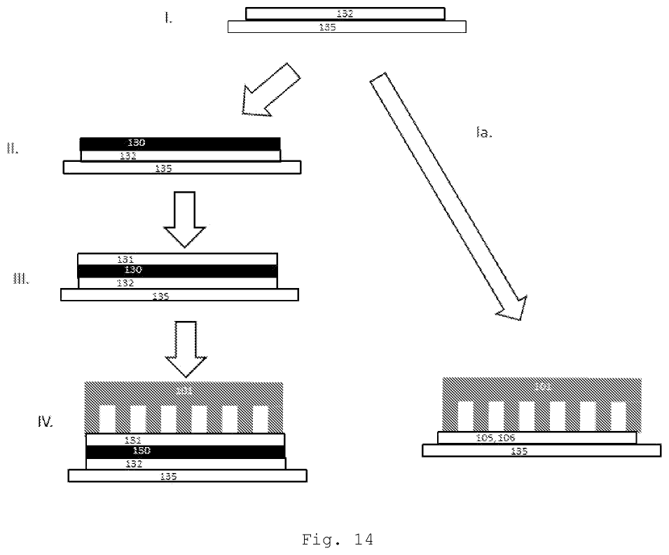

FIG. 14 shows an embodiment of the production process for the sticking system;

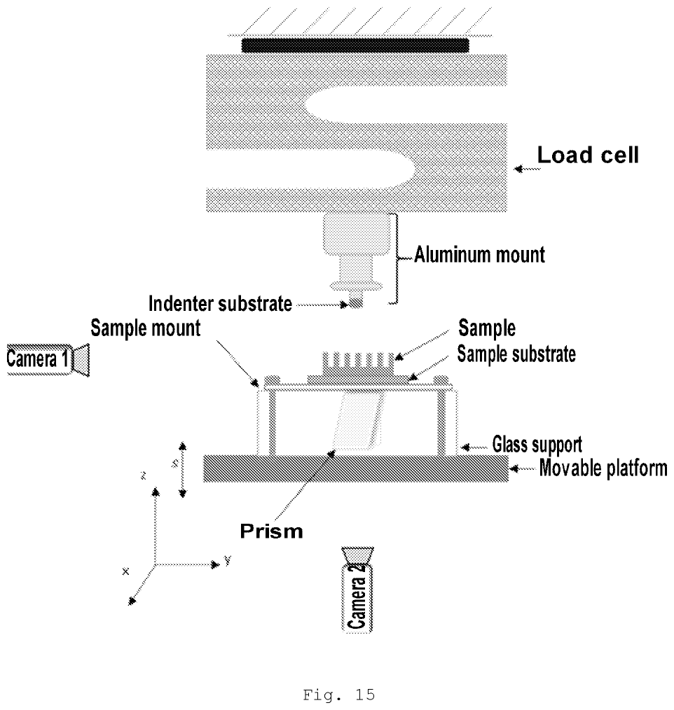

FIG. 15 shows a schematic construction of the measuring apparatus used for determining the adhesion values;

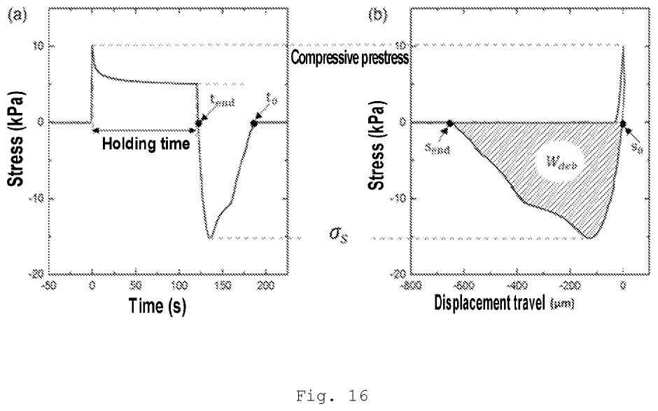

FIG. 16 shows an exemplary representation of a stress-time curve (left) and of a stress-displacement travel curve;

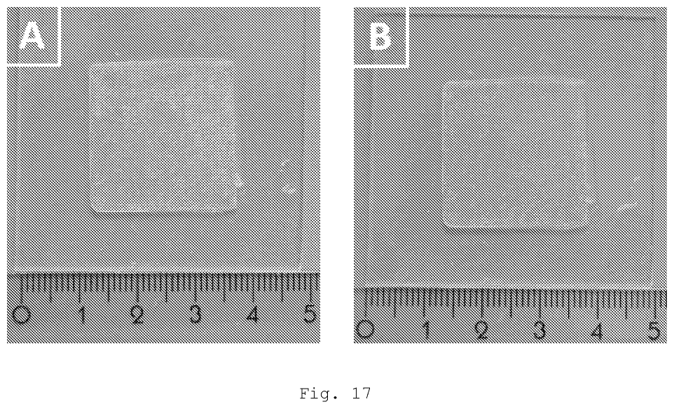

FIG. 17 shows a picture of a microstructure after extraction from the mold (A) and after mechanical treatment (B);

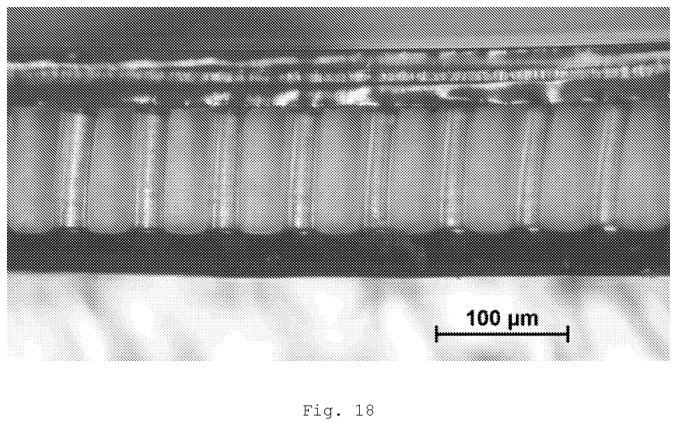

FIG. 18 shows a light-micrograph of an embodiment of the invention;

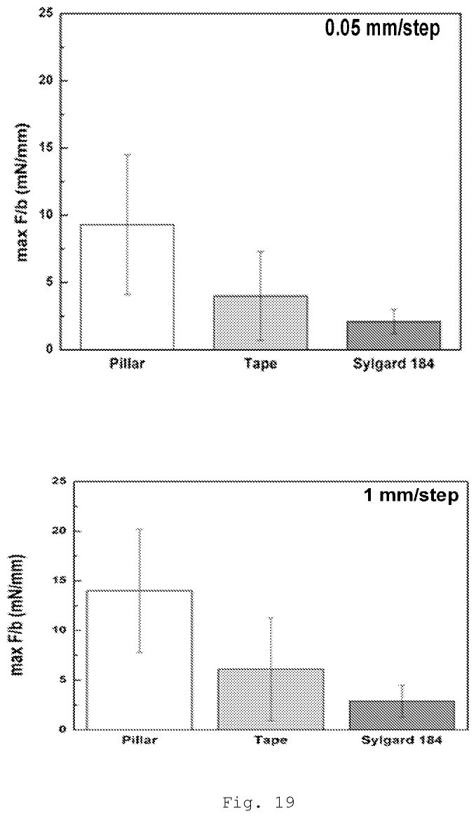

FIG. 19 shows peel measurements with different removal velocities;

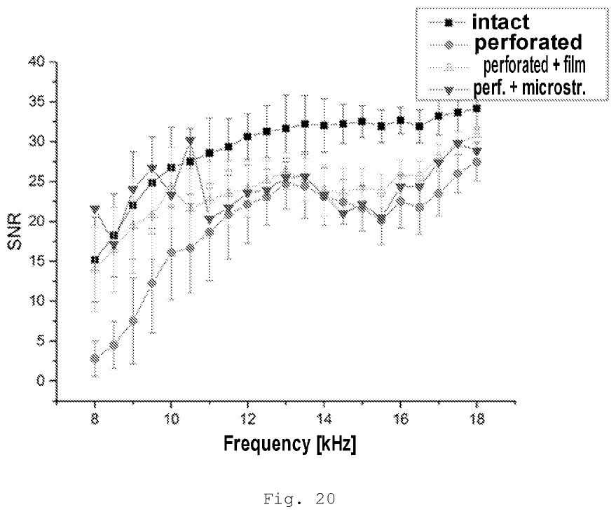

FIG. 20 shows measurement of the vibration properties in eardrums of mice.

FIG. 1 shows an overview of the operation of producing the film-terminated sticking structures. The completed sticking system consists of a microstructured part (101), made of Silastic MDX4-4210, and a terminal film, consisting here of a combination of the layers of MDX4-4210 (102, 103, step III. a.i.) and subsequent application of the skin-adhesive terminal layer of MG7-1010 (104, VI. a.i.). The terminal layer may also be produced without an MDX4 supporting layer, as shown in III. b.i. The individual steps are described below. The material and the thickness of the respective layers or structures may be varied by varying the materials or the conditions of application.

I. Wafer Modeling

The wafer (silicon wafer) is placed in a petri dish, which is filled with the material for the microstructure mold (PDMS, Elastosil 4601, Wacker, Riemerling, Germany, 100). After degassing, a glass plate (111) is placed on and curing takes place at least for 3 hours at 75° C. The cured mold (100) is then removed. The wafer has the later microstructure.

The mold produced was silanized with fluorosilane (tridecafluoro-1,1,2,2-tetrahydrooctyl)trichlorosilane, 50 μL solution) under reduced pressure (20 mbar).

II. Production of the Microstructured Part of the Sticking System

For the material of the microstructure, the two components (Silastic MDX4-4210) are weighed out and mixed in a ratio A:B (10:1). This material was used for all the structures and layers of Silastic MDX4-4210.

The mold (100) is placed onto a glass plate (111) and filled with the material for the microstructure. The surface is leveled by spincoating (3000 rpm, 120 seconds). This gives a filled mold with a small overlaying. It may be necessary to carry out degassing prior to spincoating.

In parallel the material for the backing layer (Silastic MDX4-4210) is applied to a plasma-activated glass plate. A layer having a defined thickness is produced by spincoating (9000 rpm, 120 seconds). The plasma-activated glass plate thus coated is then applied to the filled microstructure. The structure is rotated by 180° and placed onto the plasma-activated glass plate (112, oxygen-argon plasma, 2 minutes) and cured (95° C., 1 hour). This causes the microstructure to connect to the backing layer. Effective binding of the structure to the glass plate has been achieved using oxygen-argon plasma, for effective parting of the cured microstructure from the mold.

The structure is applied to a new glass plate (111). It may be necessary to align the pillars of the microstructure by mechanical action, e.g., brushing or combing (FIG. 17). This gives the A sample, i.e., the microstructure without terminating film. The separate production of the backing layer allows its thickness and material to be easily adapted.

FIG. 2 shows micrographs (A, B, C) and a schematic representation of the A sample. The microstructure was also used for the other experiments.

As a reference sample, a film composed of the same material and with similar thickness is produced via a doctor blade.

III. a.i. Production of the Supporting Layer

The material of the outer layer (Silastic MDX4-4210, 103) is applied to a glass plate (111) and distributed by spincoating (9000 rpm, 180 s). The coating is cured for an hour at 95° C. Thereafter the material for the supporting layer (Silastic MDX4-4210, 102) is applied and is distributed by spincoating (9000 rpm, 180 s). After that, the microstructure (101) produced, with the pillars, is placed onto the as yet uncured applied layer, so that the pillars at least make contact with the last-applied layer. Thereafter the whole is cured for an hour at 95° C. The structure obtained (B sample) is rotated by 180° and applied with the backing layer to a glass plate.

FIG. 3 shows micrographs of the B sample.

For the reference sample, the material of the reference structure (Silastic MDX4-4210) is applied to a glass plate, using a doctor blade, for example. The thickness is similar to the microstructure. Applied to this layer is the material of the bottom layer (Silastic MDX4-4210), which is distributed by spincoating (9000 rpm, 180 s), and the whole is cured for an hour at 95° C. The material for the second layer (Silastic MDX4-4210) is applied to this layer, distributed by spincoating (9000 rpm, 180 s), and cured for an hour at 95° C.

III. b.i. Production of the Terminal Film without Supporting Layer

The production is represented schematically in FIG. 10. The material for an auxiliary layer (120, 20% PVA polyvinyl acetate in H₂O) is applied to a glass plate (111), distributed by spincoating (3000 rpm, 60 s), and cured for 10 minutes at 95° C. Applied to this is the material of the adhesion layer (106, Dow Corning MG7-1010), which is distributed by spincoating (4000 rpm, 120 s, 100 rpm/s) and cured for an hour at 95° C. Thereafter the material for a further adhesion layer (105, Dow Corning MG7-1010) is applied and is distributed by spincoating (9000 rpm, 180 s). After that the microstructure produced (101), with the pillars, is placed onto the as yet uncured applied layer (105), so that the pillars at least make contact with the layer. Thereafter the whole is cured for an hour at 95° C. The sample is subsequently cut to size as and when necessary. Thereafter the auxiliary layer (120) is selectively dissolved with water (ultrasound bath for 10-20 minutes). The composite structure detached is applied with the backing layer to a glass plate and dried. This gives the B-OS sample. The thickness of the adhesion layer was on average 27 μm. A B-OS sample with 70 μm thickness was also produced.

For the reference sample, the material of the reference structure (Silastic MDX4-4210) is applied to a glass plate, using a doctor blade, for example. The thickness is similar to the microstructure. Applied to this layer is the material of the bottom layer (Dow Corning MG7-1010), which is distributed by spincoating (1000 rpm, 120 s), and the whole is cured for an hour at 95° C. Applied to this layer is the material for the second layer (Dow Corning MG7-1010), which is distributed by spincoating (9000 rpm, 180 s) and cured for an hour at 95° C.

The process with the auxiliary layer may also be used for producing the C sample, if the microstructure with terminating film is placed on.

IV a.i. Production of the Final Adhesion Layer

For a viscoelastic layer, a mixture of the viscoelastic material MG7-1010 (Dow Corning, Midland, USA) was prepared. The two-component system was weighed out and mixed in a ratio of 1:1.

The material for the adhesion layer (104, Dow Corning MG7-1010) is applied to the structure from III. a.i., distributed by spincoating (4000 rpm, 120 s), and cured for an hour at 95° C. This gives the C sample.

FIGS. 4, 5 and 6 show pictures of different samples. Measurement was carried out using a C sample with the following values: backing layer: 71.99+/−25.16 μm, microstructure height 208.44+/−18.87 μm, supporting layer thickness (102, 103): 19.7+/−4.94 μm, adhesion layer: 21.25+/−12.05 μm.

Table 1 and also FIG. 8 show the sticking stress and work of various samples (FIG. 7) determined in a tack test on a substrate which models the roughness of skin: determination of the sticking stress and detachment energy of the various microstructured samples in comparison to unstructured samples having a comparable layer construction. It is clearly apparent that the microstructured samples have not only a higher sticking stress but also a higher work on the rough substrate.

Table 4 shows the measured sticking stress (holding time 1 second) for various samples on substrates with different roughness (R_{z}) in kPa. Table 3 shows the same data, with the value for the smooth substrate being set at 100% in each case. It is clearly apparent that the samples having an adhesion layer (C, BoS) lose much less adhesion in the case of the rough substrates. The samples were produced with auxiliary layer or release liner and therefore have better adhesion values than the samples in table 1, since with these processes the planarity of the surface of the adhesion layer is better.

FIG. 11 shows a sticking system with partable terminal film. This system consists of the two components, the terminal film (I) and the microstructured part (II, 101), which are produced separately from one another and brought together by pressing in step 1. The layer construction of the three-layer terminal film is as follows: adhesion layer (131, Dow Corning MG7-1010), elastic supporting layer (132, Silastic MDX4-4210) and adhesion layer (132, Dow Corning MG7-1010). In the second step the sticking system can be used and can be applied to a rough surface (134, e.g., skin). In the course of use, the lowermost layer 132 becomes soiled. Since the connection between the microstructure 101 and the inner adhesion layer 131 is reversible, the microstructure and the film can be parted from one another. In this case the terminal film is disposed of, while the microstructured component can be passed back to the product life cycle. It is also possible for the terminal film to be applied to a microstructure already bearing an applied supporting layer. In the case of this process, the microstructure, which is costly and complicated to produce, can be reused.

FIG. 12 shows an exemplary embodiment of a sticking system with partable film. The film was produced by three-fold spincoating of the various materials. The terminal film (A) was produced from adhesion layers (131, 132), and a supporting layer (130). B) shows a light-micrograph of a cross section of the film. The two adhesion layers (MG7-1010) appear darker, while the middle supporting layer (MDX4-4210) appears lighter. It has a thickness of 32.32 μm. The film itself is applied to glass. This film was applied to different structures (C, microstructure of Sylgard 184, Tesafilm, Sylgard 184 film with the thickness of the microstructure) and used for the peel measurements (D, see FIG. 13, 180°, 1 mm/step, maximum force measured divided by the width of the sample). It is clearly evident that this system enables the advantages of the system of the invention, while at the same time the film remains detachable.

FIG. 18 shows a light-micrograph of the detachable film (top) on a microstructure.

FIG. 19 shows the maximum force measured for different backing systems applied to the film. The pillar is the microstructure composed of Sylgard 184 (height of the protrusions: 187±1.5 μm, backing layer 62±4 μm), the tape is Tesafilm (thickness 59±1.3 μm); Sylgard 184 is a film of Sylgard 184 (thickness 295±8.4 μm).

In the measurement with a removal velocity of 0.5 mm/step (top), measurement was carried out with a film having the following construction: MG7-1010: 30±4.5 μm/MDX4-4210: 25±5 μm/MG7-1010: 33±7 μm. Measurement was carried out three times.

In the measurement with a removal velocity of 1 mm/step (bottom), measurement was carried out with a film having the following construction: MG7-1010: 28±3.5 μm/MDX4-4210: 22±4.5 μm/MG7-1010: 27±4 μm. Measurement was carried out three times.

FIG. 13 shows a schematic representation of the peel measurement. A backing 143 is applied to a hexapod 144. The substrate 142 is applied to a perpendicular area. The substrate used had an elasticity similar to that of skin. Additionally, a modeling of artificial skin (Vitroskin) was made in order to obtain a replica of human skin. The substrate under test is mounted on a strip 141, which is connected to a load cell 140, which can be pulled away parallel to the surface, with measurement of the force. Measurement parameters used were as follows: holding time: 60 s; removal direction 180°, removal velocity 1 mm/step, preload: 1.1 kPa (area 0.75×0.75 cm). Different substrates were measured. The measurements shown in the diagrams were carried out using a model (Turboflex) of Vitroskin (Rₐ=4.43 μm, R_{z}=25.3 μm). The width of the strip was 6.5-7 mm. The measurement length was dependent on the substrate and was not more than 7 mm.

FIG. 14 shows a further embodiment of the process for producing the sticking system. In this case the adhesion layer 132, which is later to be the outermost layer, is applied to a release liner (fluorinated, 135, step I, 3M Scotchpak 9709 release liner film, fluorosilicone-coated polyester film). On this basis it is then possible to apply further layers in accordance with the desired embodiment, such as adhesion layers and supporting layers, for example, and then the microstructure is applied to these layers. The layers may be produced as in the process already described, by spincoating and curing. For application of the microstructure 101, the last applied layer with applied microstructure is cured or the last layer applied is an adhesion layer. FIG. 14 shows as step II the application of a supporting layer 130. Applied to this layer is an adhesion layer 131 (step III). Applied to this layer is the microstructure 101 (step IV). In the alternative Ia, the microstructure 101 is applied directly or after application of a further adhesion layer (105, 106). In the case of different materials, it may be necessary to treat the surface with air plasma before application of the next material. In this way it is possible to prevent soft layers in particular having their properties altered by the successive curing steps.

The release liner 135 allows the sticking system to be detached easily and without damage. There is also a shortening of the production time and the quality of the system.

The process with the release liner may also be used for producing the B sample, if the microstructure with terminating film is placed on. An alternative possibility is to apply one or more MDX4-4210 layers as the last layer, which then, as described above, are connected to the microstructure. For better attachment of the MDX4-4210 layer, it may be necessary to carry out a plasma treatment (air plasma) prior to application in order to improve the attachment.

Through the use of the release liner it has been possible to achieve a more uniform surface of the sample, resulting in a further improvement in the adhesion. For a one-second holding time, BoS sample (30 μm thickness of the adhesion layer with the same microstructure) provides a detachment energy of 641±79 mJ/m² and a stress of 14.84±1.18 kPa, while the reference gives only 79.03±39.91 mJ/m² and 7.25±3.04 kPa. If the holding time is increased, the detachment energy rises by more than two fold for the BOS sample, more specifically by 56%. The sticking stress shows an increase by 35%. In the case of the BoS-Ref sample, it is possible to measure an increase in the detachment energy of 61% and in the sticking stress of 33%.

The rheometric data were measured by means of a rheometer (MCR 300, Anton Paar formerly Physica, Graz, Austria). The rheometer has a cone-plate geometry. Before the measurements could be carried out, small amounts of the polymer mixtures were prepared in each case. MG7-1010, MDX4-4210, Sylgard 184 in a mixing ratio of 10:1 and Sylgard 184 in a mixing ratio of 100:1.6 were tested. The latter two mixtures are comparative mixtures, which are used in the literature for microstructure. Each sample was subjected to measurement three times, and was prepared freshly each time for this purpose.

FIG. 9 shows the graphical evaluation of the rheometry measurements (A: storage modulus (G′), B: complex modulus (G*), C: loss modulus (G″), D: attenuation factor (tan δ=G″/G′)).

The modulus of elasticity can be estimated for each material with the aid of the storage modulus. These values differ from the values measured with a nanoindenter, but do give the relative proportions.

On the assumption of E˜3*G′, the values reported in table 2 are obtained for 1 Hz. These values also show that Sylgard 184 10:1 is much harder than MDX4-4210. This corresponds to the measured nanoindenter values of 2.7 MPa and 1.9 MPa, respectively (steel hemisphere, sample thickness >1 mm, sample indentation depth 5000 nm).

FIG. 15 shows a schematic construction of the measuring apparatus for determining the adhesion values. In the graphic, s describes the position of the platform in the z direction. The platform moves in a positive z direction to bring sample and substrate into contact. As soon as a defined compressive prestress has been reached, the position is held for a defined holding time. The measured variables, such as the forces induced, are detected by means of a load cell and can be read off from a screen. The sample is secured by means of a bonding substrate on a glass slide, which is secured on the platform with a screw apparatus of the sample mount. In order to vary the sample position, the platform together with the sample can also be displaced in x and y directions. The position and the contact of the sample may be observed and adjusted by means of optical elements, such as the prism, camera 1 and 2.

The platform was moved toward the substrate in a positive z direction with a speed of 30 μm/s until the compressive prestress established was 70±20 mN (or 10±4 kPa). After contact between sample and substrate had been maintained for a defined holding time of either one or thirty seconds, the sample was detached from the substrate. For this the platform was moved in a negative z direction at a removal velocity of 10 μm/s. The measurement setup includes a load cell (max. 3N, Tedea-Huntleigh 1004, Vishay Precision Group, Basingstoke, GB), which is oriented for the capture of low detachment forces. The system recorded the induced normal forces F in z direction relative to the time t and to the platform position s_{z}. A prism was integrated into the sample mount for the optical detection of the sample position and hence to enable observation of the contact between sample and substrate. With the aid of two cameras (camera 1 and 2) (DMK23UX236, The Imaging Source, Germany), this made it possible to follow and record the measurements on a computer screen. A goniometer was used to adjust the contact area between sample and test substrate.

FIG. 16 shows an exemplary representation of a stress-time curve and of a stress-displacement travel curve. The respective maximum of the curves indicates the selected compressive prestress, in other words the stress with which the sample was pressed onto the test substrate. The minimum of the curves corresponds in each case to the sticking stress (σₛ). The area included by the curve in the stress-displacement travel diagram and the zero line corresponds to the detachment energy (W_{deb}) which has to be applied in order to detach the sample from the substrate. The areas of the respective test substrates were determined by optical microscopy. At the time t₀, where the detachment operation begins, but sample and substrate are still completely in contact with one another and the compressive prestress passes through the zero, the position of the platform s_{z} is referred to as s₀ (FIG. 16). The time point t_{end} is defined as the point in time at which the detachment operation was concluded (s_{end}), this being the time at which the sticking stress equals zero.

Test substrates used were as follows: model of smooth glass (polished glass) in epoxy resin (EGS area 6.2 mm², Rₐ=0.01 μm, R_{z}=0.10 μm), model of rough glass (etched matt glass) in epoxy resin (EGR, area 6.95 mm², Rₐ=0.22 μm, R_{z}=1.97 μm) and model of Vitroskin from epoxy resin (area 7.26 mm², Rₐ=9.48 μm, R_{z}=49.66 μm). Models of mouse eardrums were also used. For these models it was possible to determine a roughness depth of R_{z}=2.2 μm (pars tensa) and R_{z}=13 μm (pars flaccida). All Ra and Rz values were measured using a profilometer (SURFCOM 1500SD3, Carl Zeiss, Oberkochen, Germany). Ra and Rz were determined according to DIN EN ISO standard 4287:2010-07.

The curvature of an eardrum of pars tensa is 35.33±3.5° (determined by light microscopy). In the case of use on eardrums, however, excessively good sticking may also turn out to be disadvantageous on detachment, owing to the great sensitivity of the eardrum. In the case of the device of the invention, the adhesion is adjustable in a simple way by variation of the parameters.

FIG. 20 shows the vibration properties on mouse eardrums (intact, perforated, perforated with simple film, perforated with microstructure).

Distortion product otoacoustic emissions (DPOAE) were measured in anesthetized female mice 6-8 weeks of age. The strain was CBA/J. The frequency range investigated was from 8 kHz to 17.9 kHz. Flat films and microstructured systems having a diameter of about 1 mm were used. The diameter of the perforation was between 0.5 and 0.9 mm.

The microstructure used was a structure having an adhesion layer of 20 μm without supporting layer, protrusion height 40 μm with a diameter of 20 μm, and 20-50 μm backing layer. The smallest distance between the pillars was 20 μm. They had a regular hexagonal arrangement.

The results show that the films of the invention do not have any negative effect. For a given weight, the microstructure of the invention is somewhat bulkier than for the unstructured film.

The microstructure inherently is significantly more stable and can be applied with greater precision.

TABLE 1

| Sample | | Microstructured | | Flat | |
|---|---|---|---|---|---|
| A | Stress | 0.4 +/− 0.33 | kPa | 0 | kPa |
| sample | Detachment | 5.8 +/− 5.6 | mJ/m² | 0 | mJ/m² |
| | energy | | | | |
| B | Stress | 1.13 +/− 0.9 | kPa | 0 | kPa |
| sample | Detachment | 16.9 +/− 17 | mJ/m² | 0 | mJ/m² |
| | energy | | | | |
| C | Stress | 17 +/− 1.6 | kPa | 9.2 +/− 2.2 | kPa |
| sample | Detachment | 1003 +/− 196 | mJ/m² | 191 +/− 54 | mJ/m² |
| | energy | | | | |

TABLE 2

| | Storage modulus | E~3 * |
|---|---|---|
| | G′ [Pa] | G′ [MPa] |
| G′ Sylgard 184 10:1 | 41 400 | 1.24 |
| G′ MDX4-4210 | 360 666 | 1.08 |
| G′ MG7-1010 | 27 600 | 0.0828 |
| G′ Sylgard 100:1.6 | 7673 | 0.023 |

TABLE 3

| R_{z} [μm] | 0.10 | 1.97 | 49.66 |
|---|---|---|---|
| A | 100 | 80.7 | 2.50 |
| A-Ref | 100.00 | 56.2 | 0.00 |
| B | 100 | 66.5 | 5.23 |
| B-Ref | 100.00 | 62.9 | 0.00 |
| C | 100 | 79.9 | 32.70 |
| C-Ref | 100.00 | 67.4 | 4.80 |
| B-OS (30 μm) | 100 | 85.9 | 51.20 |
| B-OS Ref (30 μm) | 100.00 | 65.3 | 7.70 |
| B-OS (70 μm) | 100 | 83.77 | 51.30 |
| B-OS Ref (70 μm) | 100.00 | 67.2 | 12.20 |

TABLE 4

| R_{z} [μm] | 0.10 | 1.97 | 49.66 |
|---|---|---|---|
| A | 5.44 | 4.39 | 0.14 |
| A-Ref | 53.50 | 30.06 | 0.00 |
| B | 11.85 | 7.88 | 0.62 |
| B-Ref | 49.07 | 30.84 | 0.00 |
| C | 32.8 | 26.21 | 10.74 |
| C-Ref | 91.46 | 61.64 | 4.38 |
| B-OS (30 μm) | 23.86 | 20.50 | 12.22 |
| B-OS Ref (30 μm) | 80.22 | 52.40 | 6.23 |
| B-OS (70 μm) | 18.12 | 15.18 | 9.30 |
| B-OS Ref (70 μm) | 79.66 | 53.55 | 9.73 |

### REFERENCE SIGNS

100 mold for microstructure (Elastosil 4601)
101 microstructure (Silastic MDX4-4210)
102 supporting layer (Silastic MDX4-4210)
103 layer (Silastic MDX4-4210)
104 adhesion layer (Dow Corning MG7-1010)
105 adhesion layer (Dow Corning MG7-1010)
106 adhesion layer (Dow Corning MG7-1010)
110 wafer
111 glass plate
112 plasma-activated glass plate
120 auxiliary layer
130 supporting layer
131 adhesion layer
132 adhesion layer
133 soiling
134 rough surface (skin)
135 release liner
140 load cell
141 strip
142 substrate
143 backing (glass)
144 hexapod

1. A device having a structured coating, comprising:
a backing layer bearing a multiplicity of protrusions which comprise at least in each case a stem having an end face pointing away from the surface,
wherein the end face bears at least one further layer which is configured as a film, this layer comprising as surface at least one layer which has a lower modulus of elasticity than the respective protrusion.

2. The device of claim 1, wherein the protrusions have an aspect ratio of greater than 1.

3. The device of claim 1, wherein the protrusions have an aspect ratio of at least 1.5.

4. The device of claim 1, wherein the modulus of elasticity of the protrusions and of the backing layer is 1 MPa to 2.5 MPa and the modulus of elasticity of the layer having the lower modulus of elasticity is 40 kPa to 800 kPa.

5. The device of claim 1, wherein the further layer having the lower modulus of elasticity is detachable from the device.

6. The device of claim 1, wherein the device is configured for adhesion to soft substrates.

7. The device of claim 1, wherein the device is configured for adhesion to biological tissues.

8. The device of claim 7 for use in the treatment of eardrum perforations.

9. An implant comprising a device of claim 1.

10. A process for producing a device of claim 1, comprising:
applying a layer to a substrate, where the material of the layer has a different solubility than the cured materials of the device;
applying the material for the layer having the lower modulus of elasticity;
curing the layer;
optionally applying further layers;
applying the microstructure;
selectively dissolving the lowermost layer; and
detaching the device.

11. The process of claim 10, wherein instead of the lowermost layer which has a different solubility, a release liner is used.

12. A device, comprising:
a backing layer having a surface and a multiplicity of protrusions, each protrusion comprising a stem having an end face pointing away from the surface,
wherein the end face of each protrusion comprises a film, said film comprising different layers, with an outermost layer forms a surface of the film and has a lower modulus of elasticity than the protrusion.

13. A method for treating eardrum perforations, comprising:
providing the device according to claim 1; and
applying the device to a surface of the eardrum.
